(19) United States
(12) Patent Application Publication
(10) Pub. No.: US 2024/0153203 A1
(43) Pub. Date: May 9, 2024

(54) IMAGE PROCESSING METHOD, IMAGE PROCESSING DEVICE, AND PROGRAM

(71) Applicant: NIKON CORPORATION, Minato-ku, Tokyo (JP)

(72) Inventors: Yasushi TANABE, Fujisawa-shi (JP); Hiroshi KASAI, Kawasaki-shi (JP); Mariko MUKAI, Yokohama-shi (JP); Yuanting JI, Yokohama-shi (JP)

(73) Assignee: NIKON CORPORATION, Minato-ku, Tokyo (JP)

(21) Appl. No.: 18/278,128

(22) PCT Filed: Feb. 22, 2022
(86) PCT No.: PCT/JP2022/007393

§ 371 Date: Aug. 21, 2023

(30) Foreign Application Priority Data
Feb. 22, 2021 (JP) 2021-026196

(51) Int. Cl. G06T 17/00; G06T 19/00

(52) U.S. Cl. CPC G06T 17/00; G06T 19/00; G06T 2210/41

(57) ABSTRACT

Generating a three-dimensional image of a vortex vein based on OCT volume data.

An image processing method is performed by a processor and includes a step of acquiring OCT volume data including a choroid, and a step of extracting choroidal vessels based on the OCT volume data and generating a three-dimensional image of the choroidal vessels.

### TECHNICAL FIELD

Technology disclosed herein relates to an image processing method, an image processing device, and a program.

### BACKGROUND ART

U.S. patent Ser. No. 10/238,281 discloses technology for generating volume data of an examined eye using optical coherence tomography. There has hitherto been a desire to visualize blood vessels based on volume data of an examined eye.

### SUMMARY OF INVENTION

An image processing method of a first aspect of technology disclosed herein is an image processing method performed by a processor, the image processing method including: a step of acquiring OCT volume data including a choroid; and a step of extracting a choroidal vessel based on the OCT volume data and generating a three-dimensional image of the choroidal vessel.

An image processing device of a second aspect of technology disclosed herein includes a memory; and a processor connected to the memory, wherein the processor executes: a step of acquiring OCT volume data including a choroid; and a step of extracting a choroidal vessel based on the OCT volume data and generating a three-dimensional image of the choroidal vessel.

A program of a third aspect of technology disclosed herein causes a program executable by a computer to perform: a step of acquiring OCT volume data including a choroid; and a step of extracting a choroidal vessel based on the OCT volume data and generating a three-dimensional image of the choroidal vessel.

### BRIEF DESCRIPTION OF DRAWINGS

Exemplary embodiments of the present disclosure will be described in detail based on the following figures, wherein:

FIG. 1 is a schematic configuration diagram of an ophthalmic system of an exemplary embodiment;

FIG. 2 is a schematic configuration diagram of an ophthalmic device of the present exemplary embodiment;

FIG. 3 is a schematic configuration diagram of a server;

FIG. 4 is an explanatory diagram of functions implemented by an image processing program in a CPU of a server;

FIG. 5 is a flowchart illustrating image processing by a server;

FIG. 6 is a flowchart illustrating choroidal vessel three-dimensional image generation processing of step 6100 of FIG. 5;

FIG. 7 is a schematic diagram illustrating a relationship between an eyeball and positions of vortex veins;

FIG. 8 is a diagram illustrating a relationship between OCT volume data and an en face image;

FIG. 9 is a schematic diagram of vortex vein three-dimensional images;

FIG. 10 is a first example of a display screen in which a vortex vein three-dimensional image is employed;

FIG. 11 is a second example of a display screen in which a vortex vein three-dimensional image is employed;

FIG. 12 is a third example of a display screen in which a vortex vein three-dimensional image is employed;

FIG. 13 is a fourth example of a display screen in which a vortex vein three-dimensional image is employed;

FIG. 14 is a fifth example of a display screen in which a vortex vein three-dimensional image is employed; and

FIG. 15 is a sixth example of a display screen in which a vortex vein three-dimensional image is employed.

### DESCRIPTION OF EMBODIMENTS

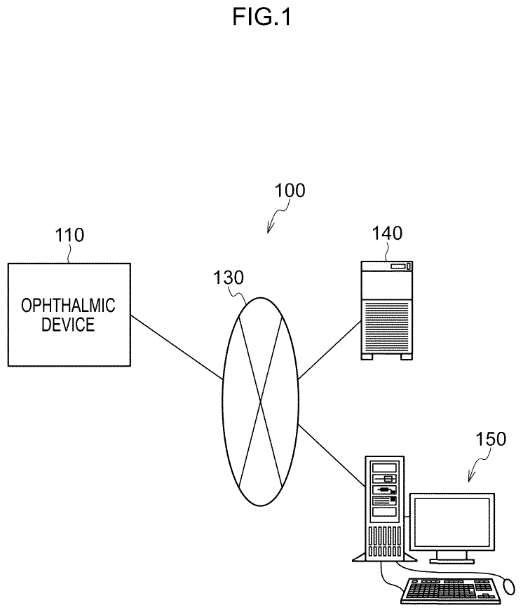

Description follows regarding an ophthalmic system 100 according to an exemplary embodiment of the present invention, with reference to the drawings. FIG. 1 illustrates a schematic configuration of the ophthalmic system 100. As illustrated in FIG. 1, the ophthalmic system 100 includes an ophthalmic device 110, a server device (hereafter referred to as “server”) 140, and a display device (hereafter referred to as “viewer”) 150. The ophthalmic device 110 acquires fundus images. The server 140 stores plural fundus images obtained by imaging a fundus of plural respective patients using the ophthalmic device 110 and eye axial lengths measured using a non-illustrated eye axial length measurement device, with these being stored associated with respective patient IDs. The viewer 150 displays fundus images and analysis results acquired by the server 140.

The server 140 serves as an example of an “image processing device” of technology disclosed herein.

The ophthalmic device 110, the server 140, and the viewer 150 are connected together through a network 130. The network 130 is a freely selected network such as a LAN, WAN, the internet, a wide area Ethernet, or the like. For example, a LAN may be employed as the network 130 in cases in which the ophthalmic system 100 is built in a single hospital.

The viewer 150 is a client in a client-server system, and plural such devices are connected together through a network. There may also be plural devices for the server 140 connected through the network in order to provide system redundancy. Alternatively, if the ophthalmic device 110 is provided with image processing functionality and with the image viewing functionality of the viewer 150, then the fundus images may be acquired and image processing and image viewing performed with the ophthalmic device 110 in a standalone state. Moreover, if the server 140 is provided with the image viewing functionality of the viewer 150, then the fundus images may be acquired and image processing and image viewing performed by a configuration of the ophthalmic device 110 and the server 140.

Note that other ophthalmic equipment (examination equipment for measuring a field of view, measuring intraocular pressure, or the like) and/or a diagnostic support device that analyzes images using artificial intelligence (AI) may be connected to the ophthalmic device 110, the server 140, and the viewer 150 over the network 130.

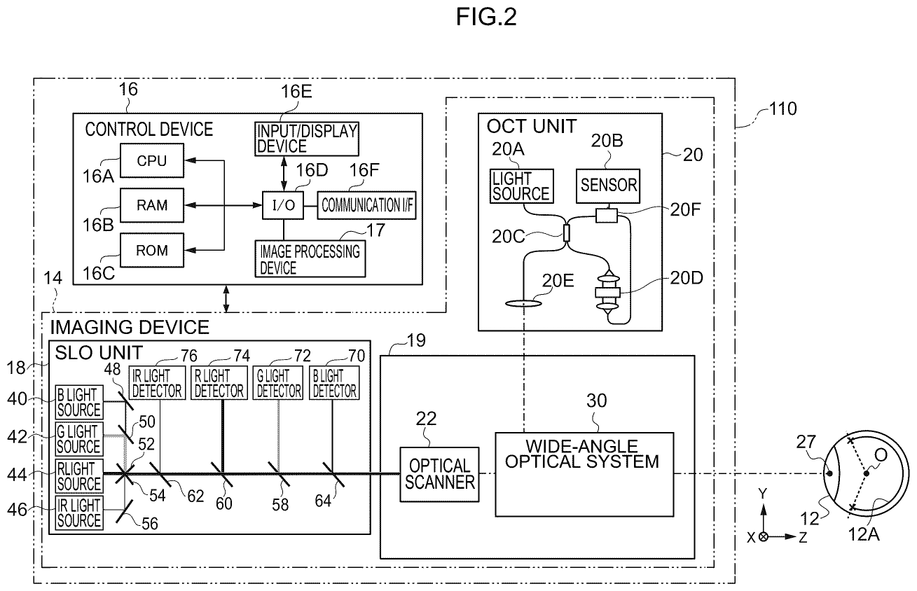

Next, explanation follows regarding a configuration of the ophthalmic device 110, with reference to FIG. 2.

For ease of explanation, scanning laser ophthalmoscope is abbreviated to SLO. Moreover, optical coherence tomography is abbreviated to OCT.

With the ophthalmic device 110 installed on a horizontal plane and a horizontal direction taken as an “X direction”, a direction perpendicular to the horizontal plane is denoted a “Y direction”, and a direction connecting the center of the pupil at the anterior eye portion of the examined eye 12 and the center of the eyeball is denoted a “Z direction”. The X direction, the Y direction, and the Z direction are thus mutually perpendicular directions.

The ophthalmic device 110 includes an imaging device 14 and a control device 16. The imaging device 14 is provided with an SLO unit 18, and an OCT unit 20, and acquires a fundus image of the fundus of the examined eye 12. Two-dimensional fundus images that have been acquired by the SLO unit 18 are referred to as SLO images. Tomographic images, face-on images (en face images) and the like of the retina created based on OCT data acquired by the OCT unit 20 are referred to as OCT images.

The control device 16 includes a computer provided with a Central Processing Unit (CPU) 16A, Random Access Memory (RAM) 16B, Read-Only Memory (ROM) 16C, and an input/output (I/O) port 16D.

The control device 16 is provided with an input/display device 16E connected to the CPU 16A through the I/O port 16D. The input/display device 16E includes a graphical user interface to display images of the examined eye 12 and to receive various instructions from a user. An example of the graphical user interface is a touch panel display.

The control device 16 is also provided with an image processing device 17 connected to the I/O port 16D. The image processing device 17 generates images of the examined eye 12 based on data acquired by the imaging device 14. Note that the control device 16 is connected to the network 130 through a communication interface 16F.

Although the control device 16 of the ophthalmic device 110 is provided with the input/display device 16E as illustrated in FIG. 2, the technology disclosed herein is not limited thereto. For example, a configuration may adopted in which the control device 16 of the ophthalmic device 110 is not provided with the input/display device 16E, and instead a separate input/display device is provided that is physically independent of the ophthalmic device 110. In such cases, the display device is provided with an image processing processor unit that operates under the control of a display control section 204 of the CPU 16A in the control device 16. Such an image processing processor unit may be configured so as to display SLO images and the like based on image signals output as instructed by the display control section 204.

The imaging device 14 operates under the control of the CPU 16A of the control device 16. The imaging device 14 includes the SLO unit 18, an imaging optical system 19, and the OCT unit 20. The imaging optical system 19 includes an optical scanner 22 and a wide-angle optical system 30.

The optical scanner 22 scans light emitted from the SLO unit 18 two dimensionally in the X direction and the Y direction. As long as the optical scanner 22 is an optical element capable of deflecting light beams, it may be configured by any out of, for example, a polygon mirror, a mirror galvanometer, or the like. A combination thereof may also be employed.

The wide-angle optical system 30 combines light from the SLO unit 18 with light from the OCT unit 20.

The wide-angle optical system 30 may be a reflection optical system employing a concave mirror such as an elliptical mirror, a refraction optical system employing a wide-angle lens, or may be a reflection-refraction optical system employing a combination of a concave mirror and a lens. Employing a wide-angle optical system that utilizes an elliptical mirror, wide-angle lens, or the like enables imaging to be performed not only of a central portion of the fundus, but also of the retina at the fundus periphery.

For a system including an elliptical mirror, a configuration may be adopted that utilizes an elliptical mirror system as disclosed in International Publication (WO) Nos. 2016/103484 or 2016/103489. The disclosures of WO Nos. 2016/103484 and 2016/103489 are incorporated in their entirety in the present specific by reference herein.

Observation of the fundus over a wide field of view (FOV) 12A is implemented by the wide-angle optical system 30. The FOV 12A refers to a range capable of being imaged by the imaging device 14. The FOV 12A may be expressed as a viewing angle. In the present exemplary embodiment the viewing angle may be defined in terms of an internal illumination angle and an external illumination angle. The external illumination angle is the angle of illumination by a light beam shone from the ophthalmic device 110 toward the examined eye 12, and is an angle of illumination defined with respect to a pupil 27. The internal illumination angle is the angle of illumination of a light beam shone onto the fundus F, and is an angle of illumination defined with respect to an eyeball center O. Correspondence relationships exists between the external illumination angle and the internal illumination angle. For example, an external illumination angle of 120° is equivalent to an internal illumination angle of about 160°. The internal illumination angle in the present exemplary embodiment is 200°.

SLO fundus images obtained by imaging at an imaging angle of view having an internal illumination angle of 160° or greater are referred to as UWF-SLO fundus images. UWF is an abbreviation of ultra-wide field (ultra-wide angled). A region extending from a posterior pole portion of a fundus of the examined eye 12 past an equatorial portion thereof can be imaged by the wide-angle optical system 30 having a field of view (FOV) angle of the fundus that is an ultra-wide field, enabling imaging of structural objects, such as vortex veins, present at fundus peripheral portions.

The ophthalmic device 110 is capable of imaging a region 12A with an internal illumination angle of 200° with respect to the eyeball center O of the examined eye 12 as a reference position. Note that an internal illumination angle of 200° corresponds to an external illumination angle of 110° with respect to the pupil of the eyeball of the examined eye 12 as the reference. Namely, the wide-angle optical system 30 illuminates laser light through the pupil at an angle of view for an external illumination angle of 110° in order to image a fundus region with an internal illumination angle of 200°.

An SLO system is realized by the control device 16, the SLO unit 18, and the imaging optical system 19 as illustrated in FIG. 2. The SLO system is provided with the wide-angle optical system 30, enabling fundus imaging over the wide FOV 12A.

The SLO unit 18 is provided with a blue (B) light source 40, a green (G) light source 42, a red (R) light source 44, an infrared (for example near infrared) (IR) light source 46, and optical systems 48, 50, 52, 54, 56 to guide the light from the light sources 40, 42, 44, 46 onto a single optical path using reflection and/or transmission. The optical systems 48, 56 are mirrors, and the optical systems 50, 52, 54 are beam splitters. B light is reflected by the optical system 48, is transmitted through the optical system 50, and is reflected by the optical system 54. G light is reflected by the optical systems 50, 54, R light is transmitted through the optical systems 52, 54, and IR light is reflected by the optical systems 52, 56. The respective lights are thereby guided onto a single optical path.

The SLO unit 18 is configured so as to be capable of switching between light sources for emitting laser light of different wavelengths or a combination of the light sources, such as a mode in which R light and G light are emitted, a mode in which infrared light is emitted, etc. Although the example in FIG. 2 includes four light sources, i.e. the B light source 40, the G light source 42, the R light source 44, and the IR light source 46, the technology disclosed herein is not limited thereto. For example, the SLO unit 18 may, furthermore, also include a white light source, in a configuration in which light is emitted in various modes, such as a mode in which G light, R light, and B light is emitted, and a mode in which white light is emitted alone.

Light introduced to the imaging optical system 19 from the SLO unit 18 is scanned in the X direction and the Y direction by the optical scanner 22. The scanning light passes through the wide-angle optical system 30 and the pupil 27 and is shone onto the fundus. Reflected light that has been reflected by the fundus passes through the wide-angle optical system 30 and the optical scanner 22 and is introduced into the SLO unit 18.

The SLO unit 18 is provided with a beam splitter 64 and a beam splitter 58. From out of the light coming from a posterior eye portion (fundus) of the examined eye 12, the B light therein is reflected by the beam splitter 64 and light other than B light therein is transmitted by the beam splitter 64. From out of the light transmitted by the beam splitter 64, the G light therein is reflected by the beam splitter 58 and light other than G light therein is transmitted by the beam splitter 58. The SLO unit 18 is further provided with a beam splitter 60 that, from out of the light transmitted through the beam splitter 58, reflects R light therein and transmits light other than R light therein. The SLO unit 18 is further provided with a beam splitter 62 that reflects IR light from out of the light transmitted through the beam splitter 60. The SLO unit 18 is further provided with a B light detector 70 to detect B light reflected by the beam splitter 64, a G light detector 72 to detect G light reflected by the beam splitter 58, an R light detector 74 to detect R light reflected by the beam splitter 60, and an IR light detector 76 to detect IR light reflected by the beam splitter 62.

Light that has passed through the wide-angle optical system 30 and the optical scanner 22 and been introduced into the SLO unit 18 (i.e. reflected light that has been reflected by the fundus) is reflected by the beam splitter 64 and photo-detected by the B light detector 70 when B light, and is reflected by the beam splitter 58 and photo-detected by the G light detector 72 when G light. When R light, the incident light is transmitted through the beam splitter 58, reflected by the beam splitter 60, and photo-detected by the R light detector 74. When IR light, the incident light is transmitted through the beam splitters 58, 60, reflected by the beam splitter 62, and photo-detected by the IR light detector 76. The image processing device 17 that operates under the control of the CPU 16A employs signals detected by the B light detector 70, the G light detector 72, the R light detector 74, and the IR light detector 76 to generate UWF-SLO images.

UWF-SLO images generated using signals detected by the B light detector 70 are called B-UWF-SLO images (blue fundus images). UWF-SLO images generated using signals detected by the G light detector 72 are called G-UWF-SLO images (green fundus images). UWF-SLO images generated using signals detected by the R light detector 74 are called R-UWF-SLO images (red fundus images). UWF-SLO images generated using signals detected by the IR light detector 76 are called IR-UWF-SLO images (IR fundus images). UWF-SLO images encompass the red fundus images, the green fundus images, the blue fundus images, and the IR fundus images. Florescent light UWF-SLO images imaged with florescent light are also encompassed therein.

The control device 16 also controls the light sources 40, 42, 44 so as to emit light at the same time. A green fundus image, a red fundus image, and a blue fundus image are obtained with mutually corresponding positions by imaging the fundus of the examined eye 12 at the same time with the B light, G light, and R light. An RGB color fundus image is obtained from the green fundus image, the red fundus image, and the blue fundus image. The control device 16 obtains a green fundus image and a red fundus image with mutually corresponding positions by controlling the light sources 42, 44 so as to emit light at the same time and imaging the fundus of the examined eye 12 at the same time with the G light and R light. An RG color fundus image is obtained from the green fundus image and the red fundus image. Moreover, a full color fundus image may be generated using the green fundus image, the red fundus image, and the blue fundus image.

A region extending from a posterior pole portion of a fundus of the examined eye 12 past an equatorial portion thereof can be imaged by the wide-angle optical system 30 with a field of view (FOV) angle of the fundus that is an ultra-wide field.

An OCT system is implemented by the control device 16, the OCT unit 20, and the imaging optical system 19 illustrated in FIG. 2. The OCT system includes the wide-angle optical system 30, and is accordingly able to perform OCT imaging of fundus peripheral portions similarly to the imaging of SLO fundus image described above. Namely, OCT imaging over a region extending from a posterior pole portion of the examined eye 12 fundus past the equatorial portion 178 is able to be performed by employing the wide-angle optical system 30 having a field of view (FOV) angle of the fundus that is an ultra-wide field. OCT data of structural objects such as vortex veins present in the fundus peripheral portions can be acquired, and tomographic images of vortex veins and a 3D structure of vortex veins can be obtained by performing image processing on the OCT data.

The OCT unit 20 includes a light source 20A, a sensor (detection element) 20B, a first light coupler 20C, a reference optical system 20D, a collimator lens 20E, and a second light coupler 20F.

Light emitted from the light source 20A is split by the first light coupler 20C. One part of the split light is collimated by the collimator lens 20E into parallel light serving as measurement light before being introduced into the imaging optical system 19. The measurement light is shone onto the fundus through the wide-angle optical system 30 and the pupil 27. Measurement light that has been reflected by the fundus passes through the wide-angle optical system 30 so as to be introduced into the OCT unit 20, then passes through the collimator lens 20E and the first light coupler 20C before being incident to the second light coupler 20F.

The other part of the light emitted from the light source 20A and split by the first light coupler 20C is introduced into the reference optical system 20D as reference light, and is made incident to the second light coupler 20F through the reference optical system 20D.

The respective lights that are incident to the second light coupler 20F, namely the measurement light reflected by the fundus and the reference light, interfere with each other in the second light coupler 20F so as to generate interference light. The interference light is photo-detected by the sensor 20B. The image processing device 17 operating under the control of an image processing section 206 generates OCT data detected by the sensor 20B. OCT images, such as tomographic images and en face images, are able to be generated in the image processing device 17 based on this OCT data.

The OCT unit 20 is able to scan a specific range (for example a rectangular range of 6 mm×6 mm) at a single time of OCT imaging. The specific range is not limited to being 6 mm×6 mm, and may be a square range of from 12 mm×12 mm, or from 23 mm×23 mm, may be a rectangular range of from 14 mm×9 mm, 6 mm×3.5 mm, or the like, and may be a freely selected rectangular range. Moreover, the specific range may be a circular range having a diameter of 6 mm, 12 mm, 23 mm, or the like.

By employing the wide-angle optical system 30, the ophthalmic device 110 is able to employ the region 12A having an internal illumination angle of 200° as the scan target. Namely, OCT imaging is performed of the specific range including vortex veins by controlling the optical scanner 22. The ophthalmic device 110 is able to generate OCT data by this OCT imaging.

Thus the ophthalmic device 110 is able to generate OCT images such as tomographic images of the fundus including vortex veins (B-SCAN images), OCT volume data including vortex veins, and en face images that are cross-sections of such OCT volume data (face-on images generated based on the OCT volume data). Note that OCT images obviously also encompass an OCT image of a fundus center portion (posterior pole portion of the eyeball where the macular, the optic nerve head, and the like are present).

The OCT data (or image data of the OCT images) is sent from the ophthalmic device 110 to the server 140 though the communication interface 16F and is stored in a storage device 254.

Note that although in the present exemplary embodiment an example is given in which the light source 20A is a wavelength swept-source OCT (SS-OCT), various types of OCT system may be employed, such as a spectral-domain OCT (SD-OCT) or a time-domain OCT (TD-OCT) system.

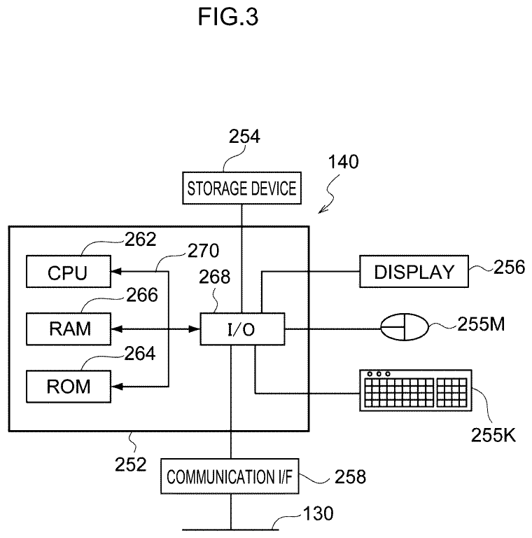

Next, description follows regarding a configuration of an electrical system of the server 140, with reference to FIG. 3. As illustrated in FIG. 3, the server 140 includes a computer main body 252. The computer main body 252 includes a CPU 262, RAM 266, ROM 264, and an input/output (I/O) port 268. The storage device 254, a display 256, a mouse 255M, a keyboard 255K, and a communication interface (I/F) 258 are connected to the input/output (I/O) port 268. The storage device 254 is, for example, configured by non-volatile memory. The input/output (I/O) port 268 is connected to the network 130 through the communication interface (I/F) 258. The server 140 is accordingly able to communicate with the ophthalmic device 110 and the viewer 150.

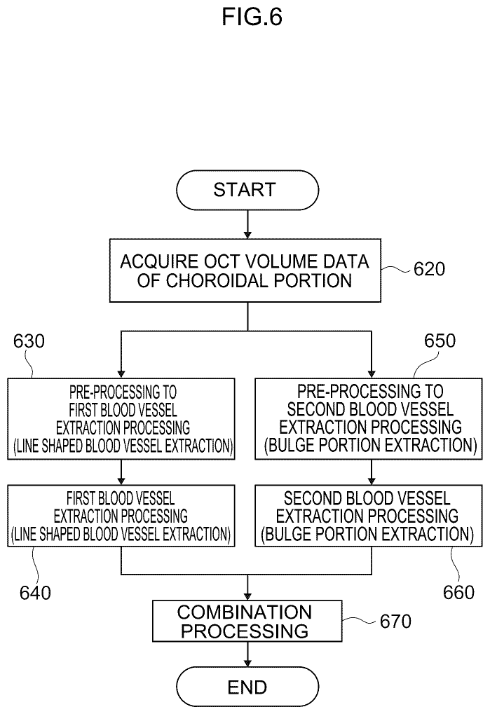

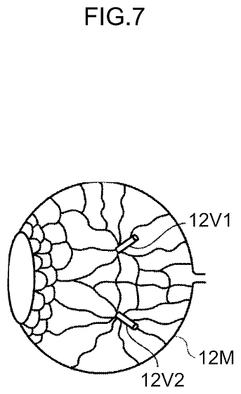

An image processing program illustrated in FIG. 6 is stored on the ROM 264 or the storage device 254.

The ROM 264 and the storage device 254 are examples of “memory” of technology disclosed herein. The CPU 262 is an example of a “processor” of technology disclosed herein. The image processing program is an example of a “program” of technology disclosed herein.

The server 140 stores respective data received from the ophthalmic device 110 in the storage device 254.

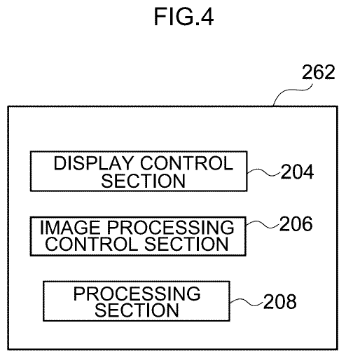

Description follows regarding various functions implemented by the CPU 262 of the server 140 executing the image processing program. The image processing program includes a display control function, an image processing function, and a processing function, as illustrated in FIG. 4. By the CPU 262 executing the image processing program including each of these functions, the CPU 262 functions as a display control section 204, the image processing section 206, and a processing section 208.

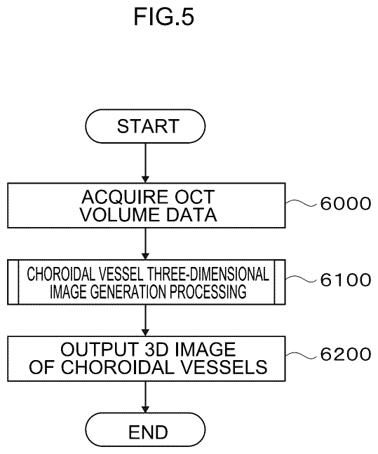

Next, explanation follows regarding a main flowchart of image processing by the server 140, with reference to FIG. 5. Image processing (an image processing method) illustrated by the flowchart in FIG. 5 is implemented by the CPU 262 of the server 140 executing the image processing program.

First, at step 6000, the image processing section 206 acquires OCT volume data including the choroid from the storage device 254.

Next at step 6100, the image processing section 206 extracts choroidal vessels based on the OCT volume data, and executes three-dimensional image generation processing (described later) to generate a three-dimensional image (3D image) of blood vessels in a vortex vein.

Then at step 6200, the processing section 208 outputs the generated three-dimensional image (3D image) of vortex vein blood vessels, and more specifically saves the generated three-dimensional image in the RAM 266 or the storage device 254, and ends the image processing.

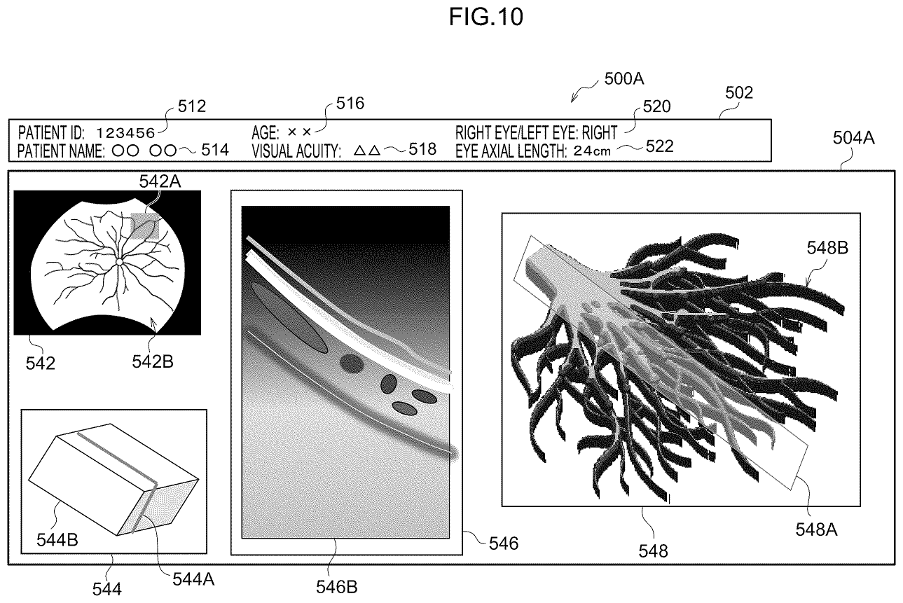

Based on instructions of the user, a display screen including a vortex vein three-dimensional image (examples of the display screen are illustrated in FIG. 10 to FIG. 15, described later) is generated by the display control section 204. The generated display screen is output to the viewer 150 as an image signal by the processing section 208. The display screen is displayed on a display of the viewer 150.

Next, detailed description follows regarding choroidal vessel three-dimensional image generation processing of step 6100, with reference to FIG. 6.

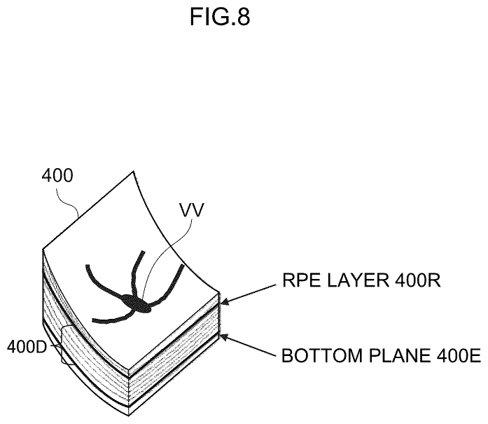

At step 620 of FIG. 6, the image processing section 206 extracts a region corresponding to the choroid from OCT volume data 400 (see FIG. 8) acquired at step 6000, and extracts (acquires) OCT volume data of a choroidal portion based on the extracted region. In the present exemplary embodiment, description follows regarding an example in which the OCT volume data 400 including a vortex vein and the choroidal vessels at the periphery of the vortex vein serves as OCT volume data 400D. In such cases the choroidal vessels indicate the vortex vein and the choroidal vessels at the periphery of the vortex vein.

More specifically, from the OCT volume data scanned so as to contain the vortex vein and the choroidal vessels at the periphery of the vortex vein, the image processing section 206 extracts the OCT volume data 400D of a lower region from a retinal pigment epithelium layer 400R (hereafter referred to as RPE layer) in the OCT volume data 400 of the region where the choroidal vessels are present.

More specifically, first the RPE layer 400R is identified by the image processing section 206 performing image processing on the OCT volume data 400 to identify boundary planes between each layer. Moreover, a layer with the highest brightness in the OCT volume data may be identified as the RPE layer 400R.

The image processing section 206 then extracts, as the OCT volume data 400D, pixel data of a region of the choroid in a region of a specific range deeper than the RPE layer 400R (a region of a specific range further away than the RPE layer when looking from a center of the eyeball). The OCT volume data of deep regions is sometimes not uniform, and so the image processing section 206 may extract, as the OCT volume data 400D, a region from the RPE layer 400R down to a bottom plane 400E obtained by the above image processing to identify boundary planes, as illustrated in FIG. 8.

The choroid region of the region of a specific range deeper than the RPE layer 400R is an example of a “choroidal portion” of technology disclosed herein.

The OCT volume data 400D for generating the choroidal vessel three-dimensional image is extracted by the processing described above.

At step 630, the image processing section 206 executes noise removal processing, and in particular speckle noise processing, as a first pre-processing to performing first blood vessel extraction processing (line shaped blood vessel extraction) on the OCT volume data 400D. This is processing to perform line shaped blood vessel extraction that excludes the effect of speckle noise and correctly reflects blood vessel shapes. Examples of speckle noise processing including Gaussian blur processing.

At the next step 640, the image processing section 206 extracts first choroidal vessels that are line shaped portions from the OCT volume data 400D by executing first blood vessel extraction processing (line shaped blood vessel extraction) on the OCT volume data 400D that has been subjected to the first pre-processing. A first three-dimensional image is generated thereby. Description follows regarding the first blood vessel extraction processing.

The image processing section 206 performs image processing using, for example, an eigenvalue filter, a Gabor filter, or the like to extract line shaped blood vessel regions from the OCT volume data 400D. The blood vessel regions in the OCT volume data 400D are pixels of low brightness (blackish pixels), and regions of contiguous low brightness pixels remain as blood vessels portions.

The image processing section 206 also performs processing on the extracted region of line shaped blood vessels to remove isolated regions not connected to surrounding blood vessels, and performs image processing such as median filtering, opening processing, erosion processing and the like to remove noise regions.

Furthermore, the image processing section 206 also performs binarization processing on the pixel data of the region of line shaped blood vessels post noise processing.

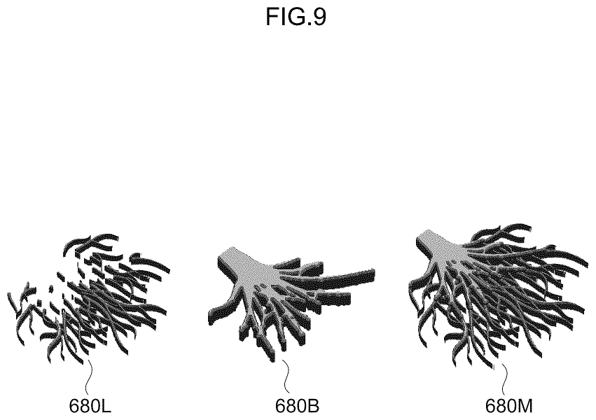

By performing the first blood vessel extraction processing as described above, only the region of line shaped blood vessels remains from the OCT volume data 400D, and a line shaped blood vessel three-dimensional image 680L as illustrated in FIG. 9 is generated. The image data of the line shaped blood vessel three-dimensional image 680L is saved in the RAM 266 by the processing section 208.

The line shaped blood vessels illustrated in FIG. 9 is an example of a “first choroidal vessels” of technology disclosed herein, and the line shaped blood vessel three-dimensional image 680L is an example of a “first three-dimensional image” of technology disclosed herein.

At step 650, the image processing section 206 executes binarization processing on the OCT volume data 400D as second pre-processing to perform second blood vessel extraction processing (bulge portion extraction) on the OCT volume data 400D. A threshold of binarization is set as a specific threshold so as to leave the blood vessel bulge portions such that in OCT volume data D the blood vessel bulge portions are black pixels and other portions are white pixels.

At step 660, the image processing section 206 extracts second choroidal vessels that are the bulge portions from the OCT volume data by removing noise regions in the binarized OCT volume data 400D. A second three-dimensional image is generated thereby. The noise regions correspond to isolated regions of regions of black pixels, and are regions corresponding to capillary blood vessels. In order to remove such noise regions, the image processing section 206 executes median filtering, opening processing, erosion processing, or the like on the binarized OCT volume data 400D so as to remove noise regions.

At step 660, in order to perform surface smoothing on the extracted bulge portions, the image processing section 206 may furthermore execute segmentation processing (image processing such as dynamic outlining, graph cut, or U-net) on the OCT volume data from which the noise regions have been removed. Reference here to “segmentation” indicates image processing to perform binarization processing on an image to be subjected to analysis so as to separate background from foreground.

By performing the second blood vessel extraction processing in this manner, a bulge portion blood vessel three-dimensional image 680B as illustrated in FIG. 9 is generated in which only a region of the bulge portion remains from the OCT volume data 400D. The image data of the bulge portion blood vessel three-dimensional image 680B is saved by the processing section 208 in the RAM 266.

The bulge portion blood vessels illustrated in FIG. 9 is an example of “second choroidal vessels” of technology disclosed herein, and the bulge portion blood vessel three-dimensional image 680B is an example of a “second three-dimensional image” of technology disclosed herein.

Either the processing of steps 630, 640 or the processing of steps 650, 660 may be executed ahead of the other processing, or they may be executed at the same time as each other.

When the processing of steps 630, 640 and the processing of steps 650, 660 have been completed, at step 670, the image processing section 206 reads the line shaped blood vessel three-dimensional image 680L and the bulge portion three-dimensional image 680B from the RAM 266. Positional alignment is then performed on these two three-dimensional images, and the line shaped blood vessel three-dimensional image 680L and the bulge portion three-dimensional image 680B are combined by computing a logical sum of the two images. A choroidal vessel three-dimensional image 680M containing a vortex vein (see FIG. 9) is generated thereby. The image data of the three-dimensional image 680M is saved by the processing section 208 in the RAM 266 and the storage device 254.

The choroidal vessel three-dimensional image 680M including the vortex vein is an example of a “choroidal vessel three-dimensional image” of technology disclosed herein.

Description follows regarding a display screen to display the generated choroidal vessel three-dimensional image (3D image) including the vortex vein. The display screen is generated by the display control section 204 of the server 140 based on instruction from the user, and is output as an image signal by the processing section 208 to the viewer 150. The viewer 150 displays the display screen on a display based on this image signal.

FIG. 10 illustrates a first display screen 500A. As illustrated in FIG. 10, the first display screen 500A includes an information area 502 and an image display area 504A.

The information area 502 includes a patient ID display field 512, a patient name display field 514, an age display field 516, a visual acuity display field 518, a right eye/left eye display field 520, and an eye axial length display field 522. Based on the information received from the server 140, the viewer 150 displays various information in each of the respective display regions from the patient ID display field 512 to the eye axial length display field 522.

The image display area 504A is an area for displaying examined eye images or the like. Each of the following display fields are provided in the image display area 504A, more specifically a UWF fundus image display field 542, an OCT volume data summary sketch display field 544, a tomographic image display field 546, and a choroidal vessel three-dimensional image display field 548.

A comment field may be provided in the image display area 504A. The comment field is a remark field enabling entry of a result of an observation by an ophthalmologist who is the user, or a freely selected diagnostic result.

A UWF-SLO fundus image 542B of the fundus of the examined eye as imaged by the ophthalmic device 110 is displayed in the UWF fundus image display field 542. A range 542A illustrating a position where the OCT volume data was acquired is displayed superimposed on the UWF-SLO fundus image 542B. In cases in which there are plural of the OCT volume data present associated with the UWF-SLO image, plural ranges may be displayed so as to be superimposed thereon, such that the user selects a single position from out of the plural ranges. FIG. 10 illustrates that a range including a top right vortex vein of the UWF-SLO image has been scanned.

An OCT volume data summary sketch (three-dimensional shape) 544B is displayed in the OCT volume data summary sketch display field 544. The user specifies a cross-section 544A desired for display using a mouse or the like so as to display, for example, a depth direction cross-section image on the OCT volume data summary sketch (three-dimensional shape). When the cross-section 544A has been specified, an en face image is generated corresponding to the cross-section 544A specified on the OCT volume data, and is displayed on the tomographic image display field 546 as a tomographic image 546B.

A choroidal vessel three-dimensional image (3D image) 548B obtained by performing image processing on the OCT volume data is displayed in the choroidal vessel three-dimensional image display field 548. This three-dimensional image 548B is a three-dimensional image that is able to be tri-axially rotated by user operation. A cross-section 548A is displayed superimposed on the choroidal vessel three-dimensional image 548B at a position corresponding to the cross-section 544A of the tomographic image 546B being displayed.

The image display area 504A of the first display screen 500A enables three-dimensional images of choroidal vessels to be perceived. Scanning a range including a vortex vein enables the vortex vein and choroidal vessels at the periphery of the vortex vein to be displayed by a three-dimensional image, enabling a user to obtain even more information for use in diagnosis.

The image display area 504A enables a position of the OCT volume data to be ascertained on an UWF-SLO image.

Furthermore, the image display area 504A enables a cross-section of the three-dimensional image to be freely selected, and enables a user to obtain detailed information about the choroidal vessels by displaying a tomographic image.

Moreover, in the three-dimensional display of the choroidal vessels by the present exemplary embodiment, three-dimensional display of choroidal vessels can be performed without employing OCT-A (OCT-angiography). This thereby enables generation of a three-dimensional image of choroidal vessels without performing complicated and high-volume calculation processing, such as obtaining a motion contrast by taking differences between OCT volume data. Although there would be a need for OCT volume data at plural different times in order to take differences in OCT-A, in the present exemplary embodiment motion contrast extraction processing is not performed, enabling generation of the choroidal vessel three-dimensional image based on a single OCT volume data.

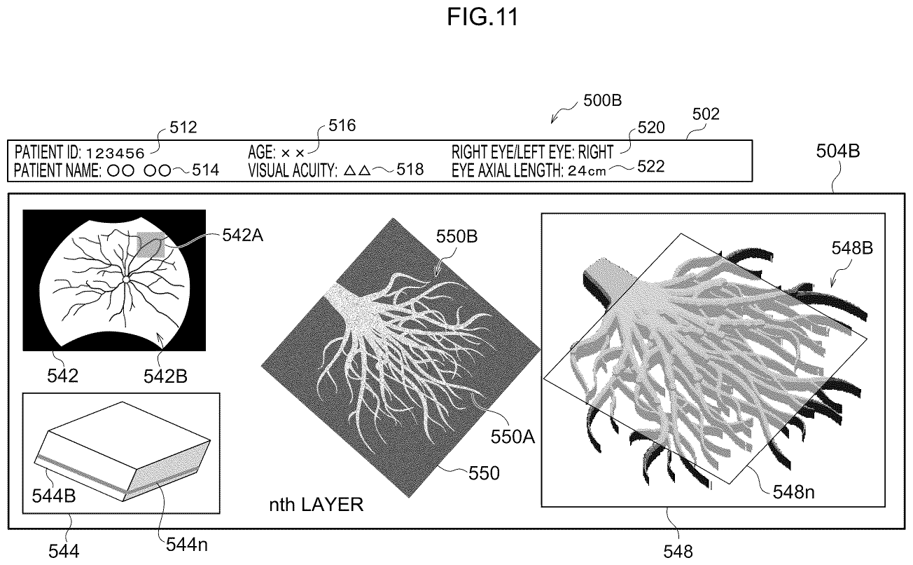

FIG. 11 illustrates a second display screen 500B. The second display screen 500B includes some of the same fields as the fields of the first display screen 500A, and so the same reference numerals are appended to the same fields and explanation thereof omitted, with differing portions described.

The second display screen 500B includes an information area 502, and an image display area 504B. The image display area 504B includes some of the same fields as the image display area 504A, and so the same reference numerals are appended to the same fields and explanation thereof omitted, with differing portions described. More specifically, the image display area 504B differs in the point that it includes an en face image display field 550 instead of the tomographic image display field 546 of the image display area 504A.

In order to display, for example, a cross-section image perpendicular to the depth direction (an en face image) in the OCT volume data summary sketch 544B, the user specifies a cross-section 544n desired for display. When the cross-section 544n has been specified, an en face image 550B corresponding to the cross-section 544n is generated based on the OCT volume data.

The en face image 550B corresponding to the cross-section 544n is displayed on the en face image display field 550. Emphasis display may be performed for the en face image 550B displayed in the en face image display field 550, such as display of an outline 550A of first blood vessels and second blood vessels extracted at steps 640, 660 of FIG. 6 in color (for example, red). Moreover, a position of the cross-section 544n may be displayed as a numerical value (e.g. the notation of “n^{th} layer” in FIG. 11).

A cross-section 548n corresponding to the cross-section 544n is displayed in the choroidal vessel three-dimensional image display field 548 superimposed on the three-dimensional image 548B.

The second display screen 500B enables a three-dimensional (3D) image of a vortex vein to be perceived at the position of the selected vortex vein. Furthermore, the second display screen 500B enables choroidal vessel three-dimensional images to be perceived. Scanning the range including the vortex vein enables display of the vortex vein and the choroidal vessels at the periphery of the vortex vein using a three-dimensional image, enabling the user to obtain more information for use in diagnosis.

Moreover, the image display area 504B enables the OCT volume data position to be ascertained on the UWF-SLO image.

Furthermore, the image display area 504B enables free selection of an enface plane of a three-dimensional image, and by displaying an en face image enables the user to obtain detailed information related to the depth direction of the choroidal vessels.

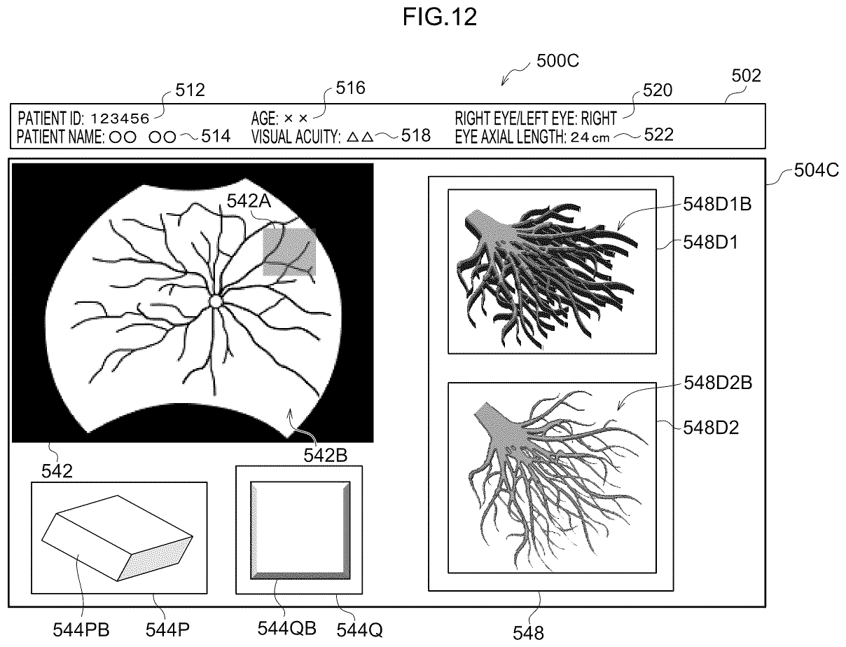

FIG. 12 illustrates a third display screen 500C. The third display screen 500C includes some of the same fields as the fields of the first display screen 500A, and so the same reference numerals are appended to the same fields and explanation thereof omitted, with differing portions described.

The third display screen 500C includes an information area 502, and an image display area 504C. The image display area 504C includes some of the same fields as the image display area 504A, and so the same reference numerals are appended to the same fields and explanation thereof omitted, with differing portions described.

More specifically, the image display area 504C also differs from the display screen 500A and 500B, and does not include the tomographic image display field 546 of the image display area 504A.

The image display area 504C differs therefrom in including two OCT volume data summary sketch display fields 544P, 544Q for perceiving the OCT volume data from two different angles (a first angle and a second angle) instead of the OCT volume data summary sketch display field 544. A summary sketch 544PB, illustrating OCT volume data depicted at an inclined angle of 45°, is displayed in the OCT volume data summary sketch display field 544P. A summary sketch 544QB, illustrating OCT volume data depicted in a state viewed from directly above, is displayed in the OCT volume data summary sketch display field 544Q. In the OCT volume data summary sketch display fields 544P, 544Q, an angle can be freely specified by operation of a user.

Furthermore, the choroidal vessel three-dimensional image display field 548 of the image display area 504C includes choroidal vessel three-dimensional image sections 548D1, 548D2 for displaying choroidal vessel three-dimensional images 548D1B, 548D2B as viewed from two different angles as identified in the OCT volume data summary sketch display fields 544P, 544Q.

The two different angles (the first angle and the second angle) may be in pre-set directions, and may be decided by AI. Note that there is no limitation to two, and there may be three or more angles.

The choroidal vessel three-dimensional images 548D1B, 548D2B may be moved in directions freely selected by the user either individually or interlocked with each other, and may be displayed enlarged in a separate widow by being clicked.

The image display area 504C of the third display screen 500C enables the user to check a choroidal vessel three-dimensional image from plural different angles. In particular, in the choroidal vessel three-dimensional image including the vortex vein, the vortex vein can be checked at an angle so as to be viewed from the sclera side.

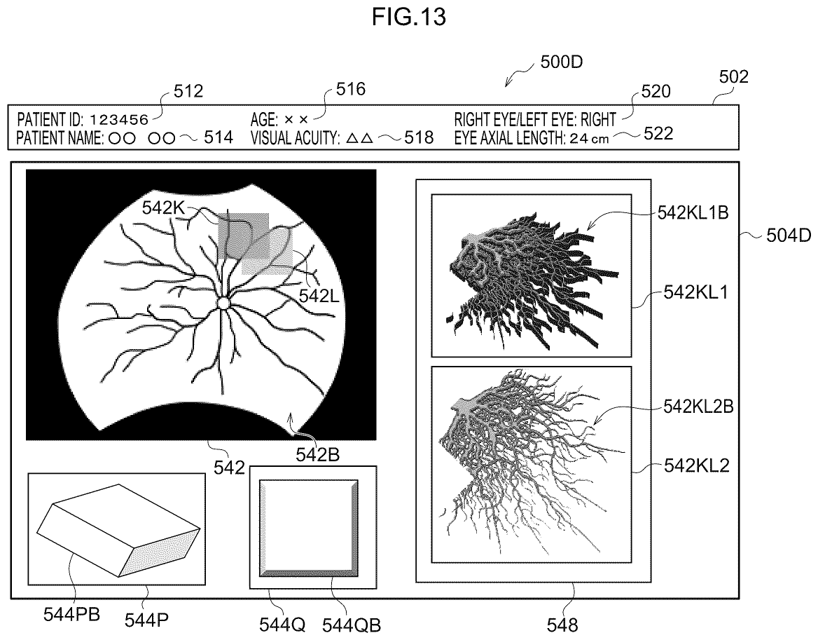

FIG. 13 illustrates a fourth display screen 500D. The fourth display screen 500D includes the some of the same fields as the fields of the third display screen 500C, and so the same reference numerals are appended to the same fields and explanation thereof omitted, with differing portions described.

The fourth display screen 500D includes an information area 502 and an image display area 504D. The image display area 504D includes some of the same fields as the image display area 504C, and so the same reference numerals are appended to the same fields and explanation thereof omitted, with differing portions described.

In order to visualize choroidal vessels of a wider range, plural OCT volume data is combined, with an example of choroidal vessel visualized using the combined OCT volume data illustrated in FIG. 13. In the UWF-SLO image display field 542, ranges where two OCT volume data, which are partly overlapping and adjacent to each other, have been acquired are displayed as ranges 542K, 542L superimposed on the UWF-SLO fundus image 542B.

The choroidal vessel three-dimensional image display field 548 of the image display area 504D includes three-dimensional image display sections 548KL1, 548KL2 for displaying three-dimensional images 548KL1B, 548KL2B obtained based on adjacent OCT volume data. The two three-dimensional images 548K1B, 548L1B of choroidal vessels are, similarly to in FIG. 12, three-dimensional images as viewed from two different angles as identified in the OCT volume data summary sketch display fields 544P, 544Q.

The image display area 504D of the fourth display screen 500D displays choroidal vessel three-dimensional images created based on plural adjacent OCT volume data. This thereby enables a choroidal vessel three-dimensional image to be checked for a wider range compared to a choroidal vessel three-dimensional image created based on single OCT volume data. This enables a user to check a choroidal vessel three-dimensional image of a wide range from plural different angles. In particular, in a choroidal vessel three-dimensional image including a vortex vein, the vortex vein can be checked at an angle so as to be viewed from the sclera side.

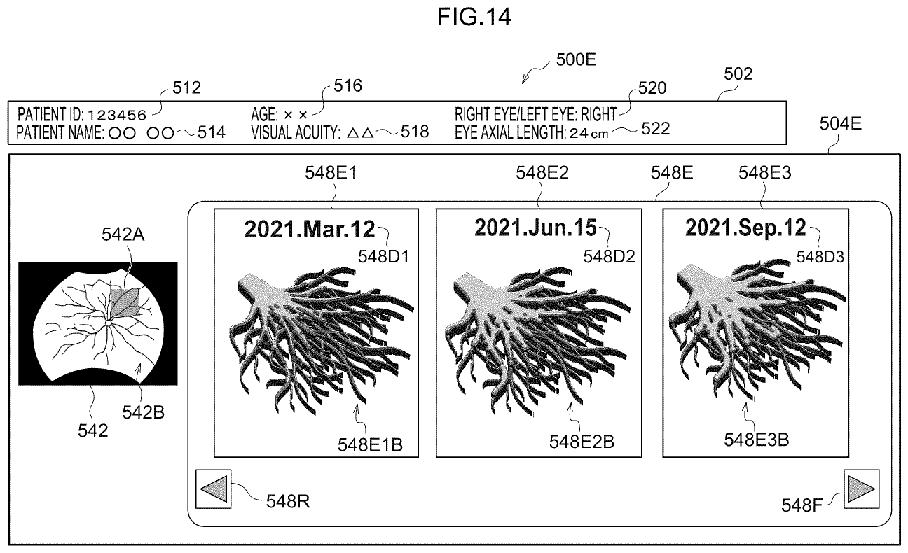

FIG. 14 illustrates a fifth display screen 500E. The fifth display screen 500E includes some of the same fields as the fields of the first display screen 500A, and so the same reference numerals are appended to the same fields and explanation thereof omitted, with differing portions described.

The fifth display screen 500E includes an information area 502 and an image display area 504E. The image display area 504E includes some of the same fields as the image display area 504A, and so the same reference numerals are appended to the same fields and explanation thereof omitted, with differing portions described.

More specifically, the image display area 504E differs in not including the OCT volume data summary sketch display field 544 and the tomographic image display field 546 of the image display area 504A. The image display area 504E also differs in including a choroidal vessel three-dimensional image display field 548E suitable for follow-up observations as described below instead of the vortex vein three-dimensional image display field 548 of the image display area 504A.

The choroidal vessel three-dimensional image display field 548E is a field for displaying a time series of plural choroidal vessel three-dimensional images from OCT volume data obtained by imaging the fundus of the same examinee at different timings.

In the example illustrated in FIG. 14, specifically the choroidal vessel three-dimensional image display field 548E includes 3 three-dimensional image display sections 548E1, 548E2, 548E3 in sequence with the oldest fundus imaging date from the left. The three-dimensional image display sections 548E1, 548E2, 548E3 include imaging date display portions 548D1, 548D2, 548D3 for displaying the fundus imaging dates. More specifically, a three-dimensional image 548E1B obtained by imaging the fundus on Mar. 12, 2021 is displayed in the three-dimensional image display section 548E1. A three-dimensional image 548E2B obtained by imaging the fundus on Jun. 15, 2021 is displayed in the three-dimensional image display section 548E2. A three-dimensional image 548E3B obtained by imaging the fundus on Sep. 12, 2021 is displayed in the three-dimensional image display section 548E3. There is no limitation to three individual three-dimensional image display fields 548E, and two or three or more three-dimensional image may be displayed.

The choroidal vessel three-dimensional image display field 548E includes a rearward button 548R to impart an instruction to display a three-dimensional image having an older imaging date than the vortex vein three-dimensional image currently being displayed, and a forward button 548F to impart an instruction to display a three-dimensional image having a newer imaging date than the three-dimensional image currently being displayed. When the rearward button 548R has been pressed, a three-dimensional image is displayed having an older imaging date than the three-dimensional image currently being displayed. When the forward button 548F has been pressed, a three-dimensional image is displayed having a newer imaging date than the three-dimensional image currently being displayed.

The fifth display screen 500E enables plural choroidal vessel three-dimensional images of an examinee to be displayed in a time series sequence. A user can accordingly check, for example, time related changes to a thickness of the vortex vein, enabling an appropriate treatment method needed at the current point in time to be confirmed.

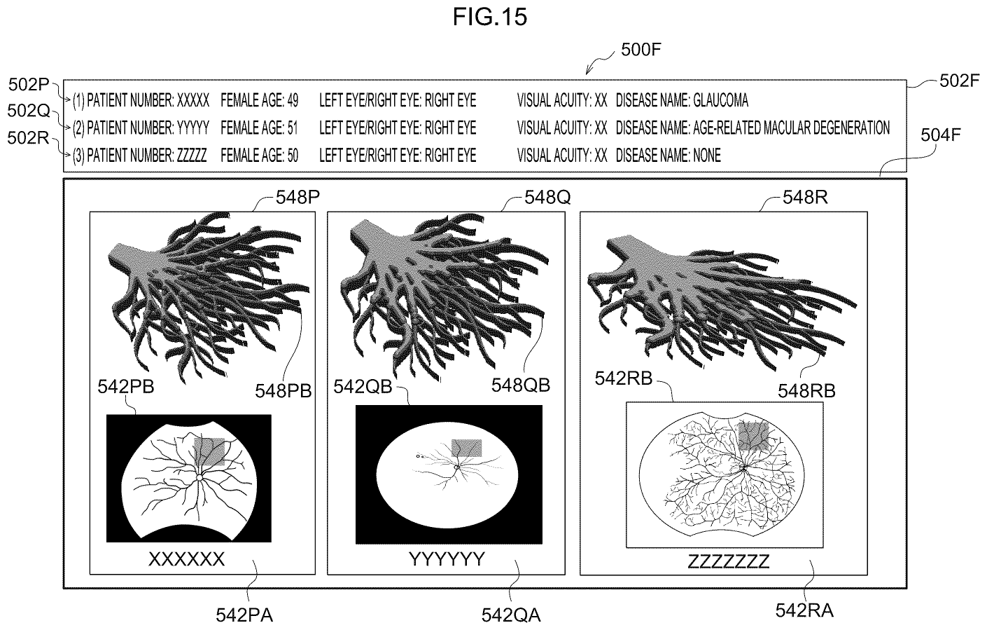

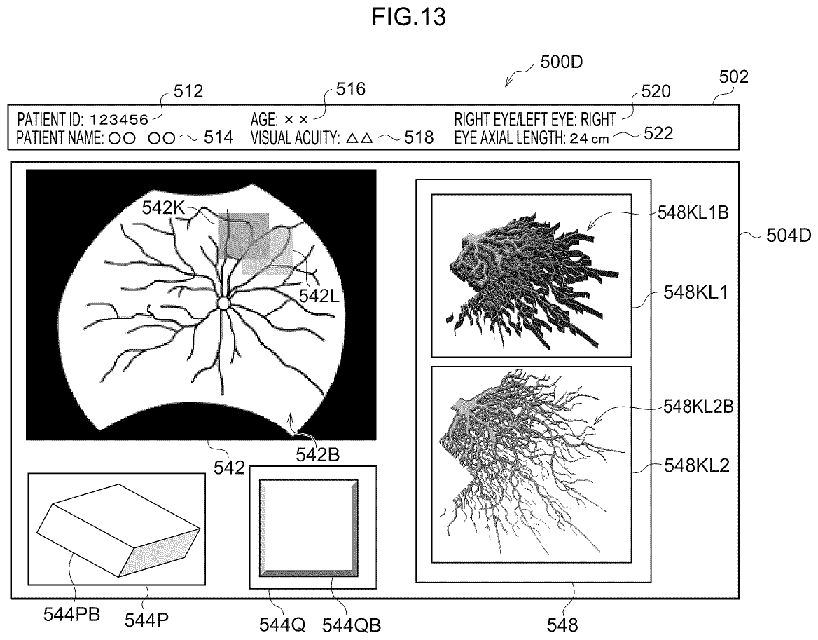

FIG. 15 illustrates a sixth display screen 500F. As illustrated in FIG. 15, the sixth display screen 500F includes an information area 502F and an image display area 504F.

The information area 502F includes patient information display fields 502P, 502Q, 502R for displaying information for plural, for example three, patients. The patient information display fields 502P, 502Q, 502R respectively include a patient number display field, a gender display field, an age display field, an age display field, a right eye/left eye display field, a visual acuity display field, and a disease name display field for each of the respective patient. A desired patient for display can be specified by the user identifying the patient ID on a non-illustrated patient identification screen. For example, patients having the same disease can be specified, or patients of the same gender and age can be specified. Three-dimensional images and UWF-SLO images corresponding to the specified patient IDs are then read by the display control section 204 of the server 140, and the sixth display screen 500F is generated.

The image display area 504F includes image display fields 548P, 548Q, 548R corresponding to each patient of the information area 502F. A patient number 542PA, a UWF-SLO image 542PB, and a choroidal vessel three-dimensional image 548PB are displayed in the image display field 548P. Although an example is illustrated in which the patient number 542PA, the UWF-SLO image 542PB, and the choroidal vessel three-dimensional image 548PB are displayed in sequence from the bottom of the page in FIG. 15, there is no limitation thereto, and the display position of each of the images may be configured so as to be changeable by user setting. Moreover, a configuration may be adopted such that, as well as the patient number 542PA, the UWF-SLO image 542PB, and the choroidal vessel three-dimensional image 548PB being displayed in the image display field 548P, attribute information and a fundus image at the same site (for example, an optic nerve head periphery, a macular periphery, or the like) for a patient the user wants to compare against may be displayed together therewith.

Similarly, a patient number 542QA, a UWF-SLO image 542QB, and a choroidal vessel three-dimensional image 548QB are displayed in the image display field 548Q. A patient number 542RA, a UWF-SLO image 542RB, and a choroidal vessel three-dimensional image 548RB are displayed in the image display field 548R.

Note that there is no limitation to images of the examined eyes of the three patients in the information area 502F, and images of the examined eyes of two patients or three or more patients may be displayed.

The sixth display screen 500F includes the respective choroidal vessel three-dimensional images of each of plural patients, and so this enables the choroidal vessel three-dimensional image of plural patients to be compared without the user switching screen.

The first display screen 500A to the sixth display screen 500F may be individually selected for display, or may be displayed in sequence.

In the present exemplary embodiment as described above the choroidal vessels are extracted based on the OCT volume data including the choroid, and the choroidal vessel three-dimensional image is generated, and so this enables the choroid to be visualized three-dimensionally.

Moreover, in the present exemplary embodiment, the choroidal vessel three-dimensional image is generated based on the OCT volume data, without employing OCT-angiography (OCT-A). The present exemplary embodiment is accordingly able to generate a choroidal vessel three-dimensional image without performing complicated high-volume calculation processing to extract a motion contrast by taking differences between OCT volume data, enabling a reduction to be achieved in the calculation volume.

In the exemplary embodiment described above, although the image processing (FIG. 5) is executed by the server 140, the technology disclosed herein is not limited thereto, and the image processing may be executed by the ophthalmic device 110, the viewer 150, or by an additional image processing device additionally provided on the network 130.

In the present disclosure, each of the configuration elements (devices and the like) may be present singly or present as two or more thereof as long as inconsistencies do not result therefrom.

Although explanation has been given in the exemplary embodiments described above regarding an example in which a computer is employed to implement image processing using a software configuration, the technology disclosed herein is not limited thereto. For example, instead of a software configuration employing a computer, the image processing may be executed solely by a hardware configuration such as a field programmable gate array (FPGA) or an application specific integrated circuit (ASIC). Alternatively, a configuration may be adopted in which some processing out of the image processing is executed by a software configuration, and the remaining processing is executed by a hardware configuration.

Thus technology disclosed herein encompasses cases in which the image processing is implemented by a software configuration utilizing a computer, and so encompasses the following technology.

First Technology

An image processing device including:
an acquisition section that acquires OCT volume data including a choroid; and
a generation section that extracts choroidal vessels based on the OCT volume data and generates a three-dimensional image of the choroidal vessels.

Second Technology

An image processing method including:
an acquisition section that performs a step of acquiring OCT volume data including a choroid; and
a generation section that performs a step of extracting choroidal vessels based on the OCT volume data and generating a three-dimensional image of the choroidal vessels.

An image processing section 206 is an example of an “acquisition section” and a “generation section” of technology disclosed herein.

The following technology may be proposed from the content disclosed above.

Third Technology

A computer program product for image processing, the computer program product including a computer-readable storage medium that is not itself a transitory signal. The computer program product is a program stored on the computer-readable storage medium, and the program causes a computer to execute a step of acquiring OCT volume data including a choroid, and a step of extracting choroidal vessels based on the OCT volume data and generating a three-dimensional image of the choroidal vessels.

A server 140 is an example of a “computer program product” of technology disclosed herein.

It must be understood that each image processing described above is merely an example thereof. Obviously redundant steps may be omitted, new steps may be added, and the processing sequence may be swapped around within a range not departing from the spirit of the technology disclosed herein.

All publications, patent applications and technical standards mentioned in the present specification are incorporated by reference in the present specification to the same extent as if each individual publication, patent application, or technical standard was specifically and individually indicated to be incorporated by reference.

The entire content of the disclosure of Japanese Patent Application No. 2021-026196 filed on Feb. 22, 2021 is incorporated by reference in the present specification.

1. An image processing method performed by a processor, the image processing method comprising:
a acquiring OCT volume data including a choroid;
a extracting a first choroidal vessel based on the OCT volume data and generating a first three-dimensional image of the first choroidal vessel;
a extracting a second choroidal vessel with a larger diameter than the first choroidal vessel based on the OCT volume data and generating a second three-dimensional image of the second choroidal vessel; and
a generating a three-dimensional image of a choroidal vessel by combining the first three-dimensional image and the second three-dimensional image.

2. The image processing method of claim 1, wherein the OCT volume data is obtained by scanning a region of a fundus including a vortex vein.

3. (canceled)

4. The image processing method of claim 1, further comprising:
a extracting choroid OCT volume data of the choroid from the OCT volume data,
wherein generating the three-dimensional image includes generating the three-dimensional image based on the choroid OCT volume data.

5. An image processing device, comprising:
a memory; and
a processor connected to the memory, wherein the processor is configured to perform processing comprising:
a acquiring OCT volume data including a choroid;
a extracting a first choroidal vessel based on the OCT volume data and generating a first three-dimensional image of the first choroidal vessel;
a extracting a second choroidal vessel with a larger diameter than the first choroidal vessel based on the OCT volume data and generating a second three-dimensional image of the second choroidal vessel; and
a generating a three-dimensional image of a choroidal vessel by combining the first three-dimensional image and the second three-dimensional image.

6. A non-transitory storage medium storing a program executable by a computer to perform:
a acquiring OCT volume data including a choroid;
a extracting a first choroidal vessel based on the OCT volume data and generating a first three-dimensional image of the first choroidal vessel;
a extracting a second choroidal vessel with a larger diameter than the first choroidal vessel based on the OCT volume data and generating a second three-dimensional image of the second choroidal vessel; and
a generating a three-dimensional image of a choroidal vessel by combining the first three-dimensional image and the second three-dimensional image.
